(19) United States
(12) Patent Application Publication
(10) Pub. No.: US 2009/0158505 A1
(43) Pub. Date: Jun. 25, 2009

(54) Urinary penis pocket for undergarments and outer garments

(76) Inventors: Sebron E. Fleming, Chester Township, PA (US); Sharon Y. Teachey, Claymont, DE (US); William Scott, Brookhaven, PA (US)

Correspondence Address: SACKET APPAREL, 2803 B PHILADELPHIA PIKE #128, CLAYMONT, DE, 19703, US

(21) Appl. No.: 11/959,507

(22) Filed: Dec. 19, 2007

(51) Int. Cl. A41B 9/02; A61F 5/44

(52) U.S. Cl. 2/405; 2/403; 604/347

(57) ABSTRACT

The leak-proof male penis pocket garment is constructed to absorb and prevent leakage of normal bodily fluids. The penis pocket is made with a leak-proof material assembled with folds and stitching. The leak-proof pocket is made with a leak-proof rubber sheeting material and a fabric that is suitable to be worn on human skin. The penis pocket garment leak-proof fabric is washable and/or suitable for dry cleaning. The penis pocket portion is made of three separate pieces of material sewn, folded and assemble in male pants and the like of such garments to form a leak-proof barrier to prevent the visible appearance on the outside of garments of accidental sprinkles, tinkles, and leaks that can occur due to urinary incontinence. The penis pocket is designed to absorb a light amount of bodily fluids and prevent seepage to the outer layers of the pocket.

### TECHNICAL FIELD OF THE INVENTION

The present invention relates to male undergarments and pants including trousers, shorts, jogging pants, shorts, pajamas, and any such male garments worn at or below the waist to have incorporate or affixed to such garments to hold the male penis thus any sprinkle, tinkle, or leaks of any bodily fluids from the male penis are maintain and/or absorbed into the pocket to prevent the soiling and embarrassing wetness on outer garments.

### DESCRIPTION OF PRIOR ART

The prior art includes various products to help men who have light incontinence challenges to over the embarrassment of soiled clothes due to sprinkles, tinkles and leaks. These products offer solutions that require the wearer to either wear a diaper and/or pad to absorb and hold the urine with no assurance that some of the flow of bodily fluids will not seep out. With the surge of contemporary and fashionable men's wear on the market there is a need for the for a product that acts as a pocket for the parking of the penis to facilitate the man on the run with a little dribble or the man on the run with a light incontinence problem that causes involuntary release of bodily fluids.

### BACKGROUND OF THE INVENTION

The present invention pertains to a male penis pocket garments for urinary incontinence to prevent leak through such as but not limited to urine, semen, or any type of fluid secretion from normal bodily fluids of the external male genital organ that can show embarrassing wetness, soiling, and discoloration of clothing.

The development of the penis pocket is a different approach to facilitate men who may experience light urinary incontinence and to aid men who want to prevent embarrassing soiling to clothes due to an occasional sprinkle, tinkle, or leak.

Many men suffer with varying types of incontinence such as: Urge Incontinence, Stress Incontinence, Overflow Incontinence, Functional Incontinence, Reflex Incontinence, Mixed Type Incontinence, and Enuresis. Incontinence can also be a result of surgical procedures such as prostate or rectal surgery.

The design allows for the parking of the penis in the pocket so as to not incur the leakage to run down the body but to be absorbed into the fabric. The absorption rate varies based on the amount of individual leakage. Any potential residue may rest in the cavity of the pocket without the potential of seepage or leak through to outer garments. Because the leak-proof fabric contains a leak-proof rubber sheeting sandwiched between layers of 100% cotton, the absorbs fluids will not pass through to the outer layers of the pocket thereby keeping the wearers body dry and the visible outer garments free from embarrassing tell-tale signs of urinary incontinence and the avoidance of soiled clothing.

### SUMMARY OF INVENTION

The intent of this product is to eliminate leakage to the external clothing surface in the area of the male genitals. The product design is to have a pocket to insert the penis inside and maintain the natural look and comfort for the personal feel of ordinary male undergarments and male pants to include said male garments which will prevent embarrassing soiling or discoloration of clothing from involuntary leakage from the male genital area. Garment and pocket are completely washable, dryer safe, and/or suitable for dry cleaning.

The design of the first layer, or outer layer, of the fabric is such that it is pleasing to the eye and the material is comfortable and pleasing to human skin. The fabric could be 100% cotton, or any percentage of cotton, ranging from about 0 to 100% of cotton to any other ratio of other material. The inner layer/sub layer can be of polyurethane, cotton-poly blends, wool, or any natural or manmade single or blended type of fabric or any combination thereof.

The usefulness of the pocket becomes apparent to those persons who have had to deal with the embarrassment of having their clothing spotted and/or stained before or after relieving themselves. By incorporating a two part cotton and one part rubber sheeting protector into the garment, such as undergarments, trousers, shorts, jogging pants, pajamas and any such male garments worn to cover the male genital area to prevent the aforesaid staining of the outer garment, such embarrassment need not occur. The second layer is a composite of two (2) parts of cotton fabric, that is absorbent, and one (1) part of rubber sheeting, sandwiched between the two (2) parts of cotton. This three (3) tiered approach, the two (2) parts of cotton sheeting on the outside of the rubber being bonded to the rubber sheeting is essential to prevent leakage to the outer garment. This protector, the cotton and rubber sheeting, is not only washable but, comfortable to the wearer.

Finally, a third layer, a layer consisting of cotton, or whichever consistency of material the outer layer may be, is sewn to the two (2) parts of cotton and rubber sheeting for pure aesthetic purposes. This material is the same material as the outer and is designed to insure that the inner garment adheres to the look and feel of the outer garment.

The folding and stitching of the material is done in a manner to prevent leaks, drips and sprinkles or accidental discharges from staining the outer garment. The outer layer is bounded to the second layer and the third inner layer is sewn to the second layer. Thus, any discharge that does occur is absorbed by the two (2) part cotton and rubber sheeting layer and will not leak out of the pocket but be absorbed into the 100% flannel fabric and not allowed to pass through to the outer garments of the wearer.

### DESCRIPTION OF THE PREFERRED EMBODIMENTS

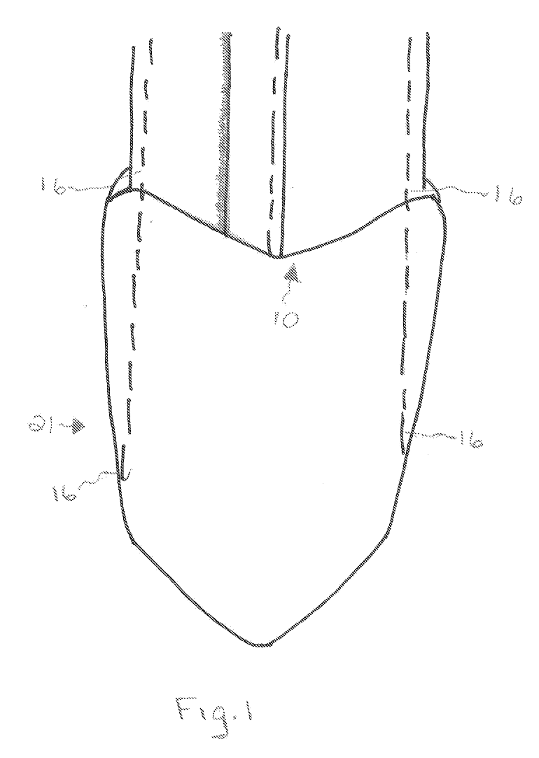

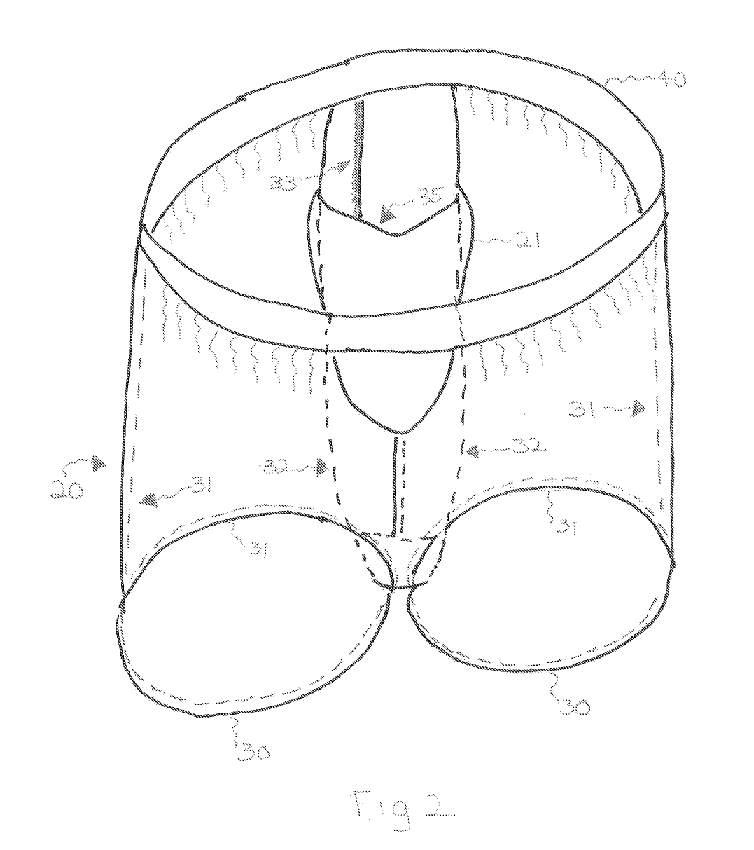

FIG. 1 is a front view of the fly-open leak proof penis pocket.

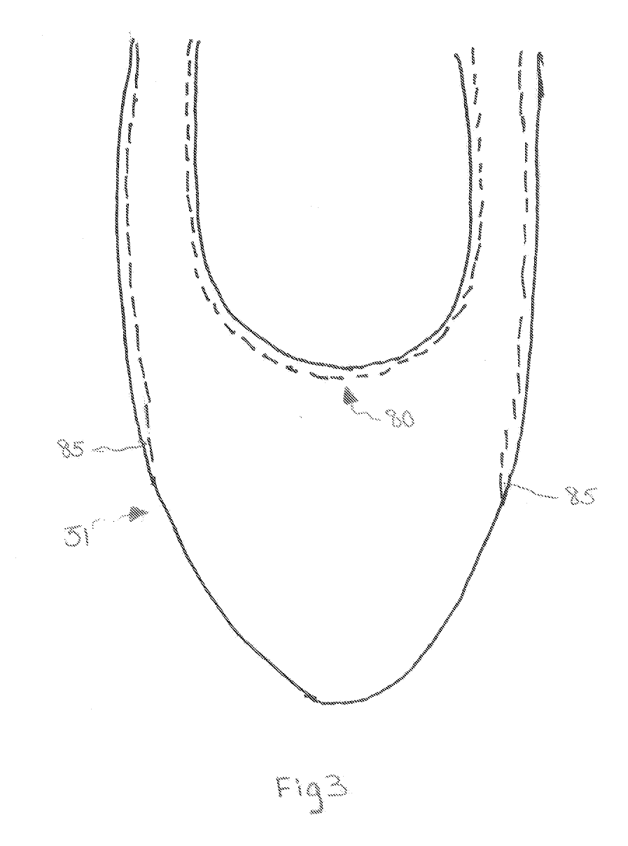

FIG. 2 is a perspective view of a male garment of this invention with the interior structure showing the penis-pocket

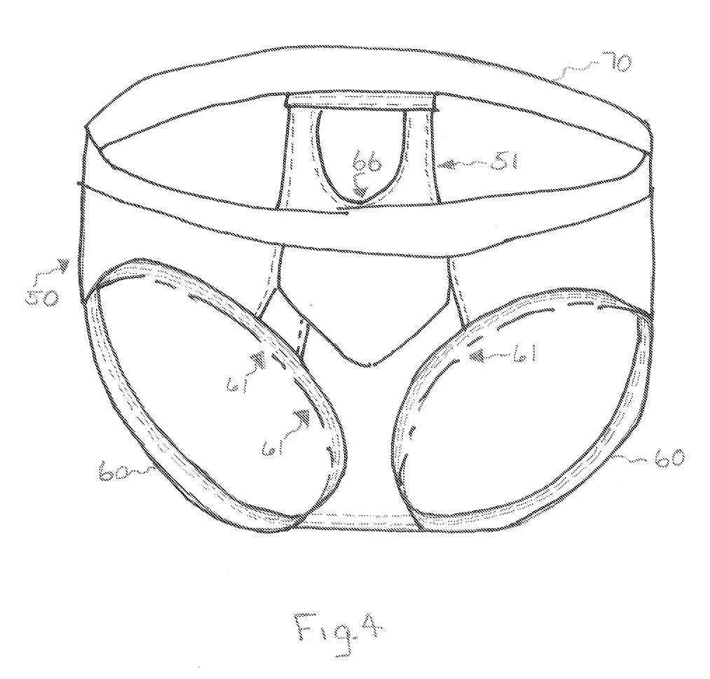

FIG. 3 is a front view of the non-fly leak proof penis pocket.

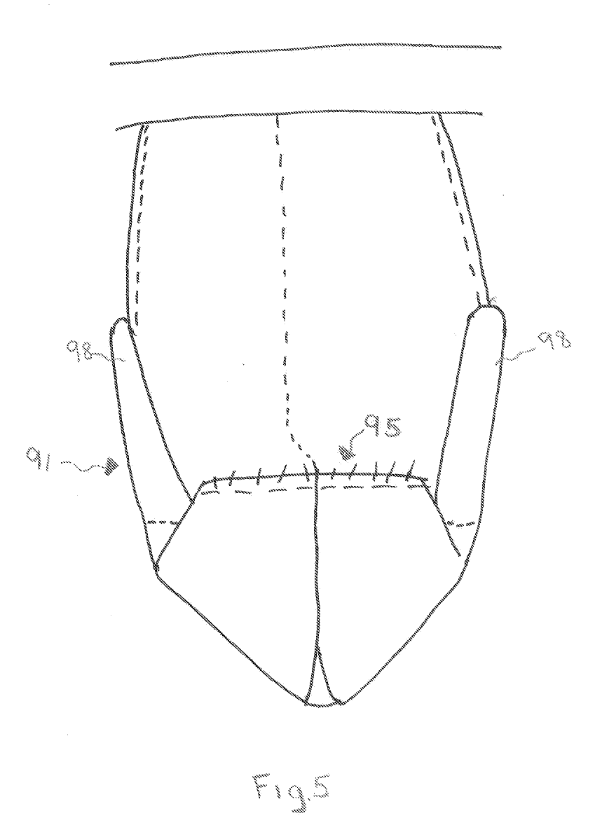

FIG. 4 is a perspective view of a male garment of this invention with the interior structure showing the penis-pocket

Referring to the accompanying drawings in which like reference numbers indicate like elements. FIGS. 1 and 3 are the base embodiment of this invention illustrated in the drawings.

Turning now to the features of a preferred embodiment of this invention illustrated in the drawings, initial reference is made to FIG. 2 from which it will be recognized that a generally conventional male undergarment, such as a boxer short 20 therein illustrated, is constructed to include an interior leak proof penis-pocket, generally indicated at 21 (see FIG. 2).

The boxer short 20 (see FIG. 2) is of generally conventional make up and incorporates a pair of like frontal panels indicated at 30 (see FIG. 2) sewn to side panels along lateral opposing edges as indicated at 31 (see FIG. 2) also sewn to back panel indicated at 32 (see FIG. 2) forming the conventional boxer shorts.

The penis-pocket is constructed with the boxer briefs using the conventional fly opening indicate at 33 (see FIG. 2) thereby rendering the same look and feel of conventional standard boxer shorts. The pocket for the placement of the penis is indicated at 35 (see FIG. 2)

The upper waist margin are joined to an elastic strip having its ends joined together to form an annular waist band, indicated generally at 40 (see FIG. 2) according to conventional practice.

In regards to FIG. 4 generally conventional male undergarment, such as briefs 50 (see FIG. 4) therein illustrated, is constructed to include an interior leak proof penis-pocket, generally indicated at 51 (see FIG. 4).

The brief 50 is of generally conventional make up and incorporates a back/side panel indicated at 60 (see FIG. 4) sewn to a two sectional (non functional aesthetics) front panels the design of the front panel is to maintain the aesthetics of standard male briefs indicated at 61 (see FIG. 4).

The penis-pocket is incorporated with the brief at the upper waist margin to an elastic strip having it ends joined together to form a waist band, indicated at 70 (see FIG. 4). The pocket for the placement of the penis is indicated at 66 (see FIG. 4)

In regards to FIG. 1. the leak-proof penis pocket is constructed using the three sections of material . One most notable is the leak-proof cotton sheeting. The open-fly penis pocket if formed with three sections (six layers based on folding) each section is of the same size and shape to form the pocket. The three sections are sewn together at all points the upper and lower and lateral sides forming one piece of garment. This garment is then folded bringing the front pocket edge midway of the entire garment, indicated at 10 (see FIG. 1). At the lower end of this fold the garment is again folded towards the back to form a V or U shaped pocket that extends upward to approximately half-way of the pocket, and fashion to the back to not pierce through the inner layer of the garment. The overlapping ends of the garment pocket are folded towards to the back and stitched to the back to not pierce through the inner layer of the garment. The front sides left and right of the pocket is also stitched to just above the back fold upper edge to not pierce through the lower portion of the pocket as indicted at 16 (FIG. 1).

In regards to FIG. 3. the leak-proof penis pocket is constructed using the three sections of material. One most notable is the leak-proof cotton sheeting. The non-fly penis pocket is formed with three sections (six layers based on folding) each section is of the same size and shape to form the pocket. The three sections are sewn together at all points the upper and lower and lateral sides forming one piece of garment. This garment is then folded bringing the front pocket edge midway of the entire garment, indicated at 80 (see FIG. 3). At the lower end of this fold the garment is again folded towards the back to form a V or U shaped pocket that extends upward to approximately half-way of the pocket, and fashion to the back to not pierce through the inner layer of the garment. The overlapping ends of the garment pocket are folded towards to the back and stitched to the back to not pierce through the inner layer of the garment. The front sides left and right of the pocket is also stitched to just above the back fold upper edge to not pierce through the lower portion of the pocket as indicted at 85 (FIG. 3).

What is claimed is:

1. The leak-proof protective pocket for the external male genital, constructed of inner and outer layers that are comfortable to the human skin and protective intermediate layers to prevent leak through to outer garments. This pocket is sewn into male pants to include but not limited to boxer shorts, trousers, pajamas, jeans, and any such male garment that has a fly opening. A first layer, third layer, fourth layer, and six layer are comfortable to human skin, the second and fifth layer are a compound material comprised of 100% cotton flannel with an intermediate layer of rubber that prevents human bodily fluids pass through to the six layer to avoid embarrassing soiling of clothes due to light male incontinence. The six layer is a two-section layer. The pocket is comprised of three sections assembled by folding and sewing pocket pieces together forming a leak-proof pocket. Wetness proof pocket integrated into male pants comprised into six layers.

2. The leak-proof Penis Pocket of claim 1, wherein layer one is comprised of fabric comfortable to the human skin being of but not limited to a cotton, cotton blend, polyester, polyester blend, silk, rayon or any such material classified safe to wear.

3. The leak-proof Penis Pocket of claim 1, wherein layer two comprises three sub-layers, the middle sub layer is rubber wherein the two outer sub-layers are 100% cotton flannel fabric will absorb light incontinence.

4. The leak-proof Penis Pocket of claim 3 wherein the middle sub-layer of layer two will prevent any pass through of bodily fluids to said layer one of claim 2 thereby keeping the wearer dry from bodily secretions.

5. The leak-proof Penis Pocket of claim 1, wherein layer three is comprised of the same material of claim 2 but not limited to say material is folded together with layer one and encasing layer two of claim 3 forming said outside of the pocket section.

6. The leak-proof Penis Pocket of claim 1, wherein layer four is a two-section layer that overlaps to form a fly opening, each section is comprised of same material of said first layer in claim 2 but not limited to said material

7. The leak-proof Penis Pocket of claim 1, wherein the layer five is a two-section layer that overlaps to form a fly opening, each section comprises three sub-layers, the middle sub layer is rubber wherein the two outer sub-layers are 100% cotton flannel fabric and will absorb light incontinence.

8. The leak-proof Penis Pocket of claim 1, wherein the middle sub-layer of layer five is a two-section layer that overlaps to form a fly opening, each section comprises three sub-layers, the middle sub layer is rubber wherein the two outer sub-layers are 100% cotton flannel fabric will absorb light incontinence of bodily fluids.

9. The leak-proof Penis Pocket of claim 1 wherein the middle sub-layer of layer five is a two-section layer that overlaps to form a fly opening, each section will prevent any pass through of bodily fluids to said layer six of claim 10 thereby protecting the wearer's garments from bodily fluid secretions.

10. The leak-proof Penis Pocket of claim 1, wherein the six layer is comprised of the same material of claim 16 but not limited to said material is sewn together with layer four and encasing layer five of claim 8 forming the said inside pocket section.

11. The leak-proof Penis Pocket of claim 1, wherein the said inside pocket section of claim 5 is joined together with sewn stitches to said outside pocket section of claim 10 forming the pocket

12. The leak-proof Penis Pocket of claim 1, wherein said pocket of claim 10 will be combined with men's boxer shorts, trousers, jeans, pajamas and any such male garment that has a fly opening but not limited to such items.

13. The leak-proof Penis Pocket of claim 1, the combining of the pocket will be consistent with men's garments of claim 12 that require a fly opening. The said pocket will be joined with sewn stitches across the top of said garments along with the elastic waist band and by center seams and/or side seams where elastic is used and affixed with and/or snaps, buttons, hook, or Velcro tape but not limited to such affixations.

14. Is a leak-proof protective pocket for the external male genital, constructed of inner and outer layers that are comfortable to the human skin and protective intermediate layers to prevent leak through to outer garments. This pocket is sewn into but not limited to such methods of affixation into male pants to include but not limited to briefs, pajamas, and jogging pants and any such male pants that do not require a fly opening. A first layer, third layer, fourth layer, and six layer are comfortable to human skin, the second and fifth layer are a compound material comprised of 100% cotton flannel with an intermediate layer of rubber that prevents human bodily fluids pass through to the six layer to avoid embarrassing soiling of clothes due to light male incontinence. The pocket is comprised of two sections assembled by sewing and folding pocket pieces together forming a leak-proof pocket. Wetness proof pocket integrated into male pants comprised into six layers

15. The leak-proof Penis Pocket of claim 14, wherein layer one is comprised of fabric comfortable to the human skin being of but not limited to a cotton, cotton blend, polyester, polyester blend, silk, rayon or any such material classified safe to wear.

16. The leak-proof Penis Pocket of claim 14, wherein the layer two comprises three sub-layers, the middle sub layer is rubber wherein the two outer sub-layers are 100% cotton flannel fabric will absorb light incontinence

17. The leak-proof Penis Pocket of claim 16 wherein the middle sub-layer of layer two will prevent any pass through of bodily fluids to said layer one of claim 15 thereby keeping the wearer dry from bodily secretions.

18. The leak-proof Penis Pocket of claim 14, wherein layer three is comprised of the same material of claim 15 but not limited to said material is sewn together with layer one and encasing layer two of claim 16 forming said outside of the pocket section.

19. The leak-proof Penis Pocket of claim 14, wherein layer four is comprised of same material of said first layer in claim 15 but not limited to said material.

20. The leak-proof Penis Pocket of claim 14, wherein the layer five comprises three sub-layers, the middle sub layer is rubber wherein the two outer sub-layers are 100% cotton flannel fabric and will absorb light incontinence.

21. The leak-proof Penis Pocket of claim 14, wherein the middle sub-layer of layer five comprises three sub-layers, the middle sub layer is rubber wherein the two outer sub-layers are 100% cotton flannel fabric will absorb light incontinence of bodily fluids.

22. The leak-proof Penis Pocket of claim 14, wherein the middle sub-layer of layer five will prevent any pass through of bodily fluids to said layer six of claim 23 thereby protecting the wearer's garments from bodily fluid secretions.

23. The leak-proof Penis Pocket of claim 14, wherein the six layer is comprised of the same material of claim 15 but not limited to said material is sewn together with layer four and encasing layer five of claim 20 forming the said inside pocket section.

24. The leak-proof Penis Pocket of claim 14, wherein the said inside pocket section of claim 23 is joined together with sewn stitches to said outside pocket section of claim 18 forming the pocket

25. The leak-proof Penis Pocket of claim 14, wherein said pocket of claim 24 will be combined with men's brief, pajamas, and jogging pants but not limited to such items.

26. The leak-proof Penis Pocket of claim 14, the combining of the pocket will be consistent with men's garments of claim 25 that do not require a fly opening. The said pocket will be joined with sewn stitches across the top of said garments along with the elastic waist band.
